(19) United States
(12) Patent Application Publication
(10) Pub. No.: US 2021/0317023 A1
(43) Pub. Date: Oct. 14, 2021

(54) HIGHLY EFFICIENT AEROBIC PHOSPHORUS-REMOVING BACTERIA CAPABLE OF SYNTHESIZING NANOPARTICLES BY MICROBIAL SELF-ASSEMBLY USING WASTE WATER

(71) Applicant: SHANDONG UNIVERSITY, Jinan (CN)

(72) Inventors: Weizhi ZHOU, Jinan (CN); Li JIANG, Jinan (CN); Zhaosong HUANG, Jinan (CN); Yanru WANG, Jinan (CN); Haixia ZHAO, Jinan (CN)

(73) Assignee: SHANDONG UNIVERSITY, Jinan (CN)

(21) Appl. No.: 17/179,449

(22) Filed: Feb. 19, 2021

Related U.S. Application Data
(63) Continuation of application No. 16/093,639, filed on Oct. 14, 2018, now abandoned.

(30) Foreign Application Priority Data
Apr. 14, 2016 (CN) 201610232204.7
Apr. 14, 2016 (CN) 201610232227.8
Apr. 14, 2016 (CN) 201610232231.4
Apr. 14, 2016 (CN) 201610236255.7
Apr. 14, 2016 (CN) 201610236259.5
Apr. 18, 2016 (CN) 201610239347.0
Apr. 18, 2016 (CN) 201610240140.5

(51) Int. Cl. C02F 3/34; C12N 1/20; C12P 3/00

(52) U.S. Cl. CPC C02F 3/341; C12N 1/20; C12R 2001/01; C12N 1/205; C12P 3/00

(57) ABSTRACT

The present application discloses a class of aerobic efficient-phosphorus-removal bacteria that enable to biologically self-assemble and synthesize nanoparticles while wastewater treatment, including Shewanella sp. CF8-6, Psychrobacter aquimaris X3-1403, and Erythrobacter citreus X3-1411. The strains in the present application have a high adaptability, which may grow, remove nutrients including phosphorus and synthesize nanoparticles within a broad range of pH values, salinity, temperatures, and nutrition concentrations of wastewater. Particularly, the outstanding performance of phosphorous removal at high-salinity has a high significance in wastewater treatment from seawater utilization such as seawater toilet-flushing to solve the fresh water resource deficiency. Self-flocculation and self-assembly are the important properties of the strains to form biofilms and synthesize calcium phosphate nanoparticles at low-concentrations, while decomposing contaminants in the wastewater. The application provides an environmental-friendly nanoparticle synthesis method with low-cost and without chemical additives, which realizes the efficient treatment of wastewater and high value phosphorous resources recovery.

### FIELD

The present application relates to the technical field of wastewater phosphorous treatment and nanomaterial preparation, and more particularly relates to a class of aerobic efficient-phosphorus-removal bacteria that enable to biologically self-assemble and synthesize nanoparticles while wastewater treatment.

### BACKGROUND

As an important element of life, phosphorous is an essential nutrient for organism growth. However, excessive discharge of phosphorous into environment could cause a series of problems, such as eutrophication and red tide, which further result in great damages to the tourism, industry, agriculture, and aquaculture. Therefore, it is urgent problem to effectively recovery phosphorous from wastewater.

Currently, sewage-treatment technologies for removing phosphorous mainly include adsorption, chemical precipitation, and biological methods. The adsorption process was mainly achieved by utilizing the affinity of some solid substances with porous and large specific surface area to phosphate radical in water. However, problems such as anti-interference, dissolution loss, and regeneration of adsorbents still exist in phosphorous removal by adsorption. Due to a relatively low adsorption capacity of conventional adsorbents, adsorption is always used as an auxiliary means to combination with other phosphorus removal methods. The chemical precipitation method produced precipitation through combination of metal cations and phosphate, which brings large amounts of chemical sludges, causing secondary pollution. Meanwhile, the expenses reagents lead to high treatment cost. And concentration of the residual metal ion is relatively high. Besides, the chemical precipitation method is not appropriate for low-phosphorous wastewater.

Compared with adsorption and chemical precipitation methods, the biological phosphorous removal method has advantages of high efficiency, low cost, and environment-friendliness. An enhanced biological phosphorous removal (EBPR) system based on the functions of polyphosphate-accumulating organisms (PAOs) is currently the most widely applied method in the biological phosphorous removal process. PAOs release phosphorous in an anaerobic condition and excessively ingest phosphorous in an aerobic condition. Finally, phosphorous removal is achieved through sludge discharge. However, phosphorous is stored in cells, and stable recycle of the phosphorous still needs further anaerobic digestion and chemical precipitation. Moreover, when treating high-salinity wastewater such as seawater toilet-flushing wastewater, a high-salinity environment would inhibit the activity of microorganisms, even with a 1% salinity. Compared with nitrifying bacteria and denitrifying bacteria, the phosphorous removal bacteria are more sensitive to salinity. It was reported that when the salinity increased from 0% to 0.4%, there was no impact on nitrogen removal, while the phosphorous removal rate dropped from 85% to 25%. Therefore, the conventional biological phosphorous removal process is greatly limited in high-salinity wastewater treatment. Meanwhile, biological sludges have drawbacks such as a long acclimatization period and difficult start-up. Thus, it has a theoretical and practical significance to screen phosphorous removal strains with a salt-tolerant property and explore their applications in further removal of phosphorous.

Nanomaterials describe materials of which at least one dimension in three-dimensional is within a nanometer range or which are comprised of nanometer elements. Due to their unique properties such as a surface effect, a small size effect, and a quantum effect, etc., nanomaterials are widely applied in industries such as energy, catalysis, biosensors, bio-medicine. Conventional physiochemical processes of synthesizing nanomaterials mainly include two-phase method, reverse micro-emulsion method, photochemical synthesis, electrode electrolysis, heating method, and ultrasonic method, etc., which have insurmountable shortcomings, for example, expensive raw materials, high energy consumption, harsh reaction conditions, or being difficult to large-scale production. Further, potentially toxic precursors and chemical reagents for a highly saturated solution was needed. All above drawbacks greatly limit applications of chemical synthesis. In contrast, the biologically synthesized methods of nanomaterials have advantages such as cleanness, mild reaction conditions, low cost, easy operation, etc, while the biosynthesized nanomaterials have good dispersiveness, stability, biocompatibility and adjustability, etc. Thus, a lot of attention is attracted by the biosynthesis of nanomaterials. The currently found microorganism species to synthesize nanomaterials are very limited, mainly including prokaryotes and eucaryon (e.g., bacteria, saccharomycete, some virus ions, fungi, and plants), which have extracellular or intracellular synthesis or nanometer self-assembly capabilities. A few reports are regarding synthesizing nanomaterials using plant extracts, natural polysaccharides, and marine polysaccharides. However, the nanomaterials synthesized by biological process are mainly focused on precious metal and metal sulfides, nanomaterials, e.g., Au, Ag, Pt, cadmium sulfide (CdS), cadmium selenide (CdSe), etc.

On the other hand, phosphorous is an important but limited resource. Excessive discharge of phosphorous will cause waste of phosphorous resources. Meanwhile, the available phosphorous resources in the land will be exhausted within future decades. Therefore, more and more attention has been paid to phosphorous recycling, especially phosphorous recovery from wastewater. Nano-hydroxyapatite (HPA), which can be applied in environment and biomedicine fields, is an effective means for recycling phosphorous. Chemical synthesis of phosphorous-containing nanomaterials was carried out in a supersaturated phosphate ion liquid with precursors. Phosphate precipitation at ambient temperature, neutral pH and in concentration below 4000 μM has not been reported. Reports about bacterial strains which can biological synthesize of calcium phosphate nanoparticles are also rare. A strain of Serratia sp. could synthesize nano-hydroxyapatite of different particle sizes and properties under different culturing conditions. This Serratia sp. strain produced calcium phosphate nanoparticles only in a highly-saturated solution (P:5 nM) and biological buffer, which is not a strictly biological synthesis, but a bio-degeneration process. Sodium glycerophosphate in matrices was decomposes by Serratia sp. through producing an atypical acid phosphatase, which released a large amount of inorganic phosphate ions to form hydroxyapatite nanoparticles with calcium ions at cell surfaces or extracellular polymers. And the formed nano-hydroxyapatite was applied to the removal of radionuclide in aqueous solution. However, the condition for Serratia sp. to form nanoparticles is still harsh. Effectively obtaining calcium phosphate nanoparticles from wastewater by a simple process is still a technical difficulty in the field.

By assembling nanomaterials into macro scale materials with a hierarchical structure, better overall collaborated property will be produced, which is an effective approach to enhance actual application capabilities of nanomaterials. In recent years, a plurality of assembly strategies has been developed, such as electrochemical precipitation, surface functionalization, and micro-imprinting technology, which have drawbacks such as highly demanding on equipment, harsh reaction conditions, prone to secondary pollution, and high cost. Therefore, developing an efficient, low-cost, and environment-friendly technology for assembling nanometer units to prepare a material with a certain structure and function is particularly significant for solving the practical applications problems of nanomaterials.

Thus, it is very important to develop a biosynthesis method that can not only degenerate pollutants in wastewater, but also synthesize and self-assemble nano-hydroxyapatite in low-concentration conditions.

### SUMMARY

To overcome the drawbacks of existing technology, an object of the present application is to provide a class of aerobic efficient-phosphorus-removal bacteria that enable to biologically self-assemble and synthesize nanoparticles while wastewater treatment.

Another object of the present application is to provide the application of the above strains in the preparation of self-assembled biomaterials.

To achieve the objects above, the present application adopts a technical proposal below:

According to a first aspect of the present application, a class of aerobic efficient-phosphorus-removal bacteria that enable to biologically self-assemble and synthesize nanoparticles while wastewater treatment is provided.

The aerobic efficient-phosphorus-removal bacteria that enable to biologically self-assemble and synthesize nanoparticles while wastewater treatment according to the present application include Shewanella sp. CF8-6, Psychrobacter aquimaris X3-1403 and Erythrobacter citreus X3-1411.

The Shewanella sp. CF8-6 was collected in China Center for Type Culture Collection on Mar. 29, 2016 at Luojiashan, Wuchang, Wuhan City, with a collection number of CCTCC M 2016154;

This strain belongs to Gram-stain-negative and may grow at the temperature ranging from 5° C. to 35° C., the pH ranging from 5.8 to 9.8, and the salinity ranging from 0˜12% in a strictly aerobic condition with a good phosphorous removal efficiency. The morphology of the bacteria cell is and observed to be bacillus with capsules and flagella under an electronic microscope. After cultured 24-hours in solid culture, the colony is characterized by round and milk white.

The Psychrobacter aquimaris X3-1403 was collected in China Center for Type Culture Collection on Mar. 29, 2016 at Luojiashan, Wuchang, Wuhan City, with a collection number of CCTCC M 2016155.

The Psychrobacter aquimaris X3-1403 in the present application belongs to Gram-stain-negative and may grow at the temperature ranging from 15° C. to 30° C. with the pH ranging from 7 to 8 and the salinity ranging from 0 to 12% (optimally 1%-5%). The morphology of the bacteria cell is observed to be coccus or Bacillus brevis with capsules but without flagellum under an electronic microscope, which may be found singly, in pairs, or in aggregations. After 24 h culturing of the strain in the LB solid culture medium, the colony is characterized by round, smooth, and cream color.

The Erythrobacter citreus X3-1411 was collected in China Center for Type Culture Collection on Mar. 29, 2016 at Luojiashan, Wuchang, Wuhan City, with a collection number of CCTCC M 2016156.

The strain belongs to Gram-stain-negative and may grow at the temperature ranging from 15° C. to 30° C. in a culturing condition where the pH value ranges from 7 to 8 and the salinity ranges from 0 to 12% (optimally 1%-5%). The morphology of the bacteria cell is observed to be bacillus with capsules but without flagella under an electronic microscope, which may be found singly, in pairs, or in short chains. After 24 h culturing of the strain in the LB solid culture medium, the colony is characterized by round, smooth, and yellow.

According to a second aspect of the present application, a microbial agent with an active ingredient selected at least one from the above mentioned bacteria (Shewanella sp. CF8-6, Psychrobacter aquimaris X3-1403, and Erythrobacter citreus X3-1411) is provided.

Further, the microbial agent may include a carrier which may be solid or liquid, which are both conventional carrier materials. The solid carrier may be selected from clay, talcum, kaolin, montmorillonite, white carbon, zeolite, siliceous rock, maizeflour cornmeal, soybean flour, polyvinyl alcohol and/or polyglycol, while the liquid carrier may be vegetable oil, mineral oil or water.

The active ingredient of the microbial agent may be the cultured living cell, a fermentation broth of the living cell, a filtrate of cell culture solution, or a mixture of cell and filtrate.

A dosage form of the microbial agent may be liquor, suspension concentrate, powder, granules, wettable powder, or water dispersible granules.

According to a third aspect of the present application, a biofilm or biofilm reactor including at least one strain of the above bacteria (Shewanella sp. CF8-6, the Psychrobacter aquimaris X3-1403, and the Erythrobacter citreus X3-1411) is provided.

The biofilm using an artificial filler or natural material as the carrier is formed by attached and flocculated Erythrobacter citreus X3-1411 on the surface of the carrier.

According to a fourth aspect of the present application, the applications of the strains, microbial agent, biofilm or biofilm reactor in phosphorous removal from wastewater are provided.

The strains or microbial agent may be used to remove phosphorous from saline wastewater or non-saline wastewater. Particularly, the strains or microbial agent is very effective in high-salinity wastewater treatment, e.g., seawater toilet-flushing wastewater. In the present application, the salinity of the high-salinity wastewater may reach 15% with a preferred salinity ranging from 0% to 10%.

According to a fifth aspect of the present application, a process of removing phosphorous from saline wastewater is provided, comprising stages of:

An above-mentioned strain is inoculated in an LB culture medium for activating. Then, the activated bacteria solution is added into a to-be-treated wastewater at a 8˜12% volume fraction.

or the microbial agent of the strain is added into the to-be-treated wastewater with the amount of 5-20 mg/L.

The utilization of the strains and/or microbial agent above in preparing a sewage treatment agent is also included in the protection scope of the present application.

According to a sixth aspect of the present application, applications of aerobic efficient-phosphorus-removal bacteria that enable to biologically self-assemble and synthesize nanoparticles while wastewater treatment or the Pseudoalteromonas sp. DSBS with a collection number of CCTCC M2013652 in preparing a nanomaterial are provided, particularly preparing a self-assembled nanomaterial in a low-phosphorous condition.

The aerobic efficient-phosphorous-removal bacteria enable to biologically self-assemble and synthesize a nanomaterial by using phosphorous of different concentrations in wastewater (including a high-phosphorus condition and a low-phosphorous condition), particularly in the low-phosphorous condition.

The low-phosphorous condition means in the low-saturated or unsaturated phosphorous concentration.

The Pseudoalteromonas sp. with the collection number of CCTCC M2013652 has been disclosed in another patent of the inventors, “Pseudoalteromonas sp. capable of efficiently removing cadmium and phosphorus in wastewater and its applications”. On this basis, the Inventors have conducted a series of extensive researches and found that the strain may not only effectively removed cadmium and phosphorous in water, but also grow in μM order or nM order unsaturated cadmium-phosphorous wastewater with low-salinity and high-salinity to form nanoparticles.

According to a seventh aspect of the present application, a biological nanomaterial synthesized and self-assembled by an above-mentioned strain is provided, wherein the biological nanomaterial is synthesized and self-assembled in a phosphorous-contained wastewater by the aerobic efficient-phosphorus-removal bacteria that enable to biologically self-assemble and synthesize nanoparticles while waste-water treatment or the Pseudoalteromonas sp. DSBS with a collection number of CCTCC M2013652.

The concentration of the phosphorous in the wastewater ranges from 0.3 mM to 1.3 mM.

According to an eighth aspect of the present application, a preparing process of a self-assembled biological nanomaterial, comprising stages of strain activating, and culturing and self-assembling of the activated strain in a phosphorous-contained wastewater, is provided.

In the preparing process, the stage of activating the strain includes: inoculating the strain into an LB culture medium, activating and culturing it for 18˜30 h at 180˜220 rpm, 15° C.˜30° C. Preferably, the condition for the activation and culture is 200 rpm, 25° C., and 24 h.

The LB culture medium includes 1% peptone and 0.3% yeast, mixed with artificial seawater.

During the preparing process, the stage of culturing and self-assembling comprises includes: inoculating the activated strain in a phosphorous-contained wastewater, and culturing it for 42˜54 h at 180˜220 rpm, 15° C.˜30° C.; preferably, culturing it for 48 h at 200 rpm, 25° C.

The inoculation of the activated strain is 8˜12% (v/v).

The stage of culturing and self-assembling further comprises: centrifuging a cultured solution to remove the supernatant, and obtaining bacteria containing nanomaterials, namely the biological nanomaterial.

The centrifuging is performed at a rotary speed of 5000 rpm for 10 min.

In the present application, the phosphorous-contained wastewater may be seawater toilet-flushing wastewater or domestic sewage or an unsaturated/low-saturated system containing cadmium and phosphorous. In the unsaturated/low-saturated system containing cadmium and phosphorous, concentrations of cadmium and phosphorous range from μM grade to nM grade.

According to a ninth aspect of the present application, utilization of the biological nanomaterials, mainly including applications in the environment field and biomedicine field, is provided.

In the environment field, the biological nanomaterials may be applied in fluorine removing, phenol adsorbing, and removing of lead, cadmium, other heavy metals and radioactive wastes.

The heavy metal which may react with sulfur atoms and nitrogen atoms on the amino acid side chain, have a high toxicity. As environment pollution incidents occurred, heavy metal pollution and remediation has gained wide attention. Existing remediation methods for heavy metal pollutions mainly include physical remediation, chemical remediation, and biological remediation. Chemical remediation requires addition of chemical agents to the polluted environment such as soil and waterbody to achieve the adsorption, redox reaction and precipitation of the heavy metal ions. Although this method is simple in operation and apparent in effect, it easily causes secondary pollution and costs dearly. Utilization of phosphate-contained materials to remedy the heavy metal pollution in environment is an effective approach. The biological nanomaterial in the present application contains nano-hydroxyapatite generated from the bacteria cells, which may be used for remedying heavy metal environment pollution with advantages such as simple operation and low cost. Besides, activated bacteria cells in the biological nanomaterial could further adsorb the heavy metals.

In the biomedicine field: the biological nanomaterials after removal of organics (retaining the nano-hydroxyapatite after removal of the organics) of the present application are used in preparing drug carriers, anti-tumor drugs, hard tissue repair materials, artificial bones and artificial teeth.

The nanomaterials have important applications in biomedicine, human health and other life science industries, such as used as a carrier to transport drugs, and for biomedical examination and diagnosis. Calcium phosphate such as hydroxyapatite, a major inorganic mineral component of bones and teeth of animals and human body, has a good activity and biocompatibility. Hydroxyapatite ceramics is a very prospective material for artificial bones and artificial teeth. The biologically synthesized calcium phosphate material not only has the properties of nanomaterials, but also has a better biocompatibility and adaptability, which ensures broad and prospective applications of the nano-hydroxyapatite in the biomedicine field.

According to a tenth aspect of the present application, a preparing process for nano-hydroxyapatite is provided, where the nano-hydroxyapatite is obtained by purifying and isolating above biological nanomaterial. The specific method of purifying and isolating is calcining the biological nanomaterial to remove the organics and then obtaining the nano-hydroxyapatite.

The nano-hydroxyapatite material obtained from the above process has a uniformly distributed particle size. And a desired particle size and morphology size may be obtained by controlling conditions of the preparing process. Besides, the nano-hydroxyapatite obtained by self-assembly of the strains in the present application has a good film-formation property and is thus widely applied in preparing thin film materials.

The present application has the following beneficial effects:

(1) The strains in the present application have an excellent environmental adaptability, which may grow in salt-free and high-salinity condition within a broad range of pH values, temperatures, and nutrition. Moreover, phosphorous in wastewater was efficiently removed by the strains to reach a compulsory discharge standard with a final concentration blow 0.5 mg/L. Particularly, the strains of the present application have a good phosphorous-removal and purification effect for high-salinity wastewater such as wastewater from toilet-flushing seawater, which has a great significance in solving fresh water deficiency and establishing an effective sea-water toilet-flushing wastewater utilization system;

(2) The phosphorous removal by the strains of the present application is easy to operate and low in cost, which is only required activation and inoculation of strains into phosphorous-contained wastewater further culturing. Therefore;

(3) Phosphorous removal was achieved under a single aerobic condition by the strains in the present application, which simplifies the phosphorous removal process and improves operability of the phosphorous removal process, providing a new approach for biological removal of phosphorous;

(4) The strains in the present application implement phosphorous removal by precipitating metal phosphate (calcium phosphate precipitation in the system of the present application) in unsaturated/low-saturated wastewater system;

(5) The strains have properties of self-flocculation and self-assembly. While decomposing pollutants in the wastewater, the strains synthesize calcium phosphate nanoparticles with raw materials in wastewater in a low-concentration. Besides, self-assembly needn't addition of chemical agents and is thus environment-friendly with a low cost. The present application realizes recycling of phosphorous resources;

(6) The preparing process of the biological nanomaterials in the present application requires a mild condition, which is easy to operate, clean and pollution-free, low in costs, efficient, and capable of large-scale dissemination and application;

(7) The biological nanomaterials prepared according to the present application have nanoparticles distributed at and surrounding bacteria cell surfaces. The biological nanomaterials have a prominently improved effect in removing fluorine, adsorbing phenol, removing lead and cadmium or other heavy metals in water and cleaning radioactive wastes. After removing the bacteria cells in the biological nanomaterials, porous nanomaterials may be formed as drug carriers.

### BRIEF DESCRIPTION OF THE DRAWINGS

FIG. 1 shows a phylogenetic tree of Strain CF8-6;

FIG. 2 shows a diagram of Psychrobacter aquimaris X3-1403 Grain-stain result;

FIG. 3 shows an AFM (Atomic Force Diagram) image of Psychrobacter aquimaris X3-1403 morphology of the bacteria cell;

FIG. 4 shows a diagram of Erythrobacter citreus X3-1411 Grain-stain result;

FIG. 5 shows an AFM image of Erythrobacter citreus X3-1411 morphology of the bacteria cell;

FIG. 6a shows a growth curve of CF8-6 under different salinities;

FIG. 6b shows phosphorous removal rates of CF8-6 under different salinities;

FIG. 7 shows effects of Psychrobacter aquimaris X3-1403 to remove TP, COD, NH₄⁺—N, and TN from simulated seawater toilet-flushing wastewater;

FIG. 8 shows effects of Erythrobacter citreus X3-1411 to remove TP, COD, NH₄⁺—N, and TN in a simulated seawater toilet-flushing wastewater;

FIG. 9 shows an image (AFM image) of a nanomaterial synthesized from the strain CF8-6 with simulated high-salinity wastewater Formulation (1);

FIGS. 10a-10f show images of nanomaterials synthesized from the strain CF8-6 with simulated high-salinity wastewater Formulation (2), wherein FIG. 10a shows an AFM image; FIG. 10b and FIG. 10c show TEM (Transmission Electron Microscopy) images of nanoparticles; FIG. 10d, FIG. 10e, and FIG. 10f show TEM images of self-assembled nanoparticles;

FIG. 11 shows an electron microscope image of self-flocculation of the strain Psychrobacter aquimaris X3-1403 in simulated seawater toilet-flushing wastewater;

FIG. 12 shows an electron microscope image of self-assembly and nanoparticle synthesis of the strain Psychrobacter aquimaris X3-1403 in simulated seawater toilet-flushing wastewater;

FIG. 13 shows an electron microscope image of the strain Erythrobacter citreus X3-1411 self-flocculated in simulated seawater toilet-flushing wastewater;

FIG. 14 shows a TEM image of nanomaterial synthesized from the strain Erythrobacter citreus X3-1411 in simulated seawater toilet-flushing wastewater;

FIGS. 15A-15E show an AFM image (A), SEM (Scanning Electron Microscope) images (B, C, D), and EDS (energy-dispersive spectrometry) analysis (E) of Pseudoalteromonas sp. DSBS to form nanoparticles in low-salinity wastewater;

FIG. 16 shows phosphorous removal of Pseudoalteromonas sp. DSBS in high-salinity wastewater; and

FIGS. 17A-17C show an AFM image (A), SEM (Scanning Electron Microscope) image (B), and EDS (energy-dispersive spectrometry) analysis (C) of the strain to form nanoparticles in high-salinity wastewater.

### DETAILED DESCRIPTION OF EMBODIMENTS

It should be noted that the detailed depictions below are all schematic, intended to provide for further explanations of the present application. Unless otherwise indicated, all technical and scientific terms used herein have the same meanings as generally understood by those of normal skill in the art.

It needs to be noted that the terms used here are only for describing preferred embodiments, not to limit the present application with the exemplary embodiments. Unless otherwise explicitly indicated, a singular form is also intended to include a plural form; besides, it should also be understood that when terms “include” and/or “comprise” are used in the present specification, they indicate presence of the features, steps, operations, devices, components, and/or their combinations.

To make those skilled in the art understand the technical solutions of the present application more clearly, the technical solution of the present application will be described in more detail with reference to the preferred embodiments.

Testing materials used in the embodiments of the present application are all conventional testing materials in the art, which may be purchased from commercial channels.

The bacterial strains Shewanella sp. CF8-6, the Psychrobacter aquimaris X3-1403, and the Erythrobacter citreus X3-1411 were deposited in the China Center for Type Culture Collection on Mar. 29, 2016 at Luojiashan, Wuchang, Wuhan City, with the following collection numbers: CCTCC M 2016154 for the Shewanella sp. CF8-6, CCTCC M 2016155 for the Psychrobacter aquimaris X3-1403, and CCTCC M 2016156 for the Erythrobacter citreus X3-1411.
The deposits were made and accepted under the Budapest Treaty and applicants aver under 37 CFR § 1.808(a) that the deposit was made under conditions that assure that:
(1) Access to the deposit will be available during pendency of the patent application making reference to the deposit to one determined by the Director to be entitled thereto under § 1.14 and 35 U.S.C. § 122, and
(2) Subject to paragraph (b) of this section, all restrictions imposed by the depositor on the availability to the public of the deposited material will be irrevocably removed upon the granting of the patent.

### Example 1: Isolation and Identification of Efficient Phosphorous Removal Bacteria that Enable to Biologically Self-Assemble Under a Low-Phosphorous Condition

1. Isolation of Strains

(1) 464 strains from the China South Sea was cultured in a seawater LB liquid culture medium for 24 h (200 rpm, 25° C.) and kept static for 15 min to observe whether the bacteria cells are self-flocculated;

(2) The self-flocculation capable strains were centrifuged at 5000 rpm for 10 min to discard the supernatant. The bacteria cells were washed twice with deionized water, and then observed by TEM whether nano-order particles are produced on the bacteria cell surfaces;

(3) Strains that enable self-flocculation and have nano-grade particles produced on bacteria cell surfaces were screened and inoculated into simulated seawater toilet-flushing wastewater or simulated high-salinity household wastewater with a 10% inoculation amount. Samples were taken by time to measure the effects of strains in TP and COD removing from wastewater. Strains with a higher phosphorous removal speed and phosphorous removal rate were selected as target strains.

In the isolation method, the seawater LB culture medium includes 1% peptone and 0.3% yeast, mixed with artificial seawater.

Components of the simulated seawater toilet-flushing wastewater are shown in Table 1.

TABLE 1
Formula of the Simulated Seawater Toilet-Flushing
Wastewater (Mixed with Artificial Seawater):

| | Concentration | | Concentration |
|---|---|---|---|
| Component | (mg/L) | Component | (mg/L) |
| glucosum | 500 | Yeast extract | 150 |
| anhydricum | | | |
| Sodium acetate | 550 | NH₄Cl | 800 |
| Peptone | 220 | KH₂PO₄ | 180 |

Components of Simulated High-Salinity Household Sewage:

C₆H₁₂O₆.H₂O 1.5 g/L, CH₃COONa 0.75 g/L, MgSO₄.7H₂O 1.18 g/L, NH₄Cl 0.9 g/L, KH₂PO₄.2H₂O 0.066 g/L (P:10 mg/L), NaCl 30 g/L.

All culture mediums are subjected to high-temperature sterilization for 20 min at 121° C. The inoculation is carried out on a clean worktable. The strains are preserved in a 1.5 mL centrifugal tube (containing 600 uL bacteria solution and 300 uL 30% glycerol) in an ultra-low temperature freezer at −80° C. for a long term.

Through isolation and screening, 3 strains are obtained, which enable self-flocculation, and have nano-order particles produced on the bacteria cell surfaces with a high phosphorous removal speed and phosphorous removal rate, i.e., strain CF8-6, strain X3-1403, and strain X3-1411.

2. Strain Identification

The 3 strains obtained from isolation and screening are identified, specifically:

2.1 Identification of the Strain CF8-6

2.1.1 Physiological and Biochemical Characterizations:

Physiological and biochemical characterizations of the strain: the strain CF8-6 belongs to Gram-stain-negative and may grow at the temperature ranging from 5° C. to 35° C., pH ranging from 5.8 to 9.8, and salinity ranging from 0 to 12% in a strictly aerobic condition with a good phosphorous removal effect. The morphology of the bacteria cell is observed to be bacillus with capsules and flagellum by an electronic microscope. After 24-hours culture of the strain in solid culture, the colony is characterized by round and milk white.

2.1.2 Molecular Biological Identification:

Molecular Biological Identification of the Strain CF8-6:

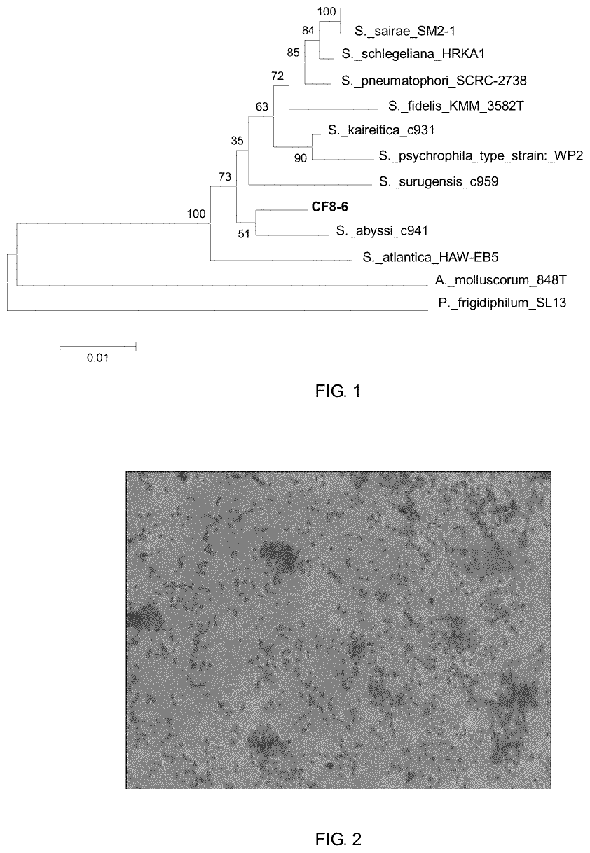

The DNA of the strain CF8-6 was extracted with a kit. The 16S rDNA sequence was expanded through PCR. And the 16S rDNA sequence of the strain CF8-6 was obtained and shown in the sequence table SEQ ID NO:1. Nucleotide homology comparison between the 16S rDNA of the strain CF8-6 and the 16S rNDA sequence recorded in the GenBank was carried out with the LBAST program to obtain that the strain CF8-6 belongs to Shewanella. Therefore, this bacterium is named as Shewanella sp. CF8-6; the phylogenetic tree of the strain is shown in FIG. 1.

the Shewanella sp. CF8-6 was collected in China Center for Type Culture Collection at Luojiashan, Wuchang, Wuhan City, with a collection number of CCTCC M 2016154;

2.2 Identification of the Strain X3-1403

2.2.1 Physiological and Biochemical Characterizations:

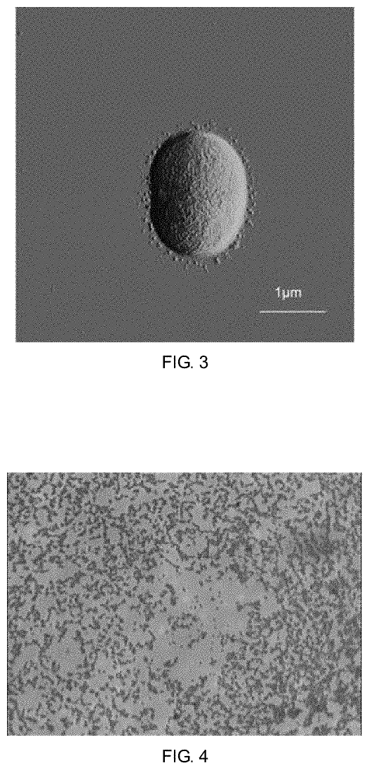

Physiological and biochemical characterizations of the strain: the strain X3-1403 belongs to Gram-stain-negative and may grow at the temperature ranging from 15° C. to 30° C. with the pH ranging from 7 to 8 and the salinity ranging from 0 to 12% (optimally 1%˜5%). The morphology of the bacteria cell is observed to be coccus or Bacillus brevis with capsules but without flagella under an electronic microscope, which may be found singly, in pairs, or in aggregation. After 24 h culturing of the strain in the LB solid culture medium, the colony is characterized by round, smooth, and cream color, as shown in FIG. 3.

2.2.2 Molecular Biological Identification:

Analysis of 16S rDNA Sequence

The sequence of the 16s rDNA of the strain X3-1403 are shown in SEQ ID No. 2. Similarity sequence comparison between the measured 16S rDNA nucleotide sequence and that recorded in the NCBI GenBank database was performed, indicating: strain X3-1403 and Psychrobacter are located at a same minimum branch, while the similarity of 16S rDNA between strain X3-1403 and Psychrobacter aquimaris is 99.64%. In conjunction with the colony morphology and 16S rDNA sequence analysis, strain X3-1403 is identified as Psychrobacter aquimaris.

The Psychrobacter aquimaris X3-1403 is collected in China Center for Type Culture Collection at Luojiashan, Wuchang, Wuhan City, with a collection number of CCTCC M 2016155.

2.3 Identification of the Strain X3-1411

2.3.1 Physiological and Biochemical Characterizations:

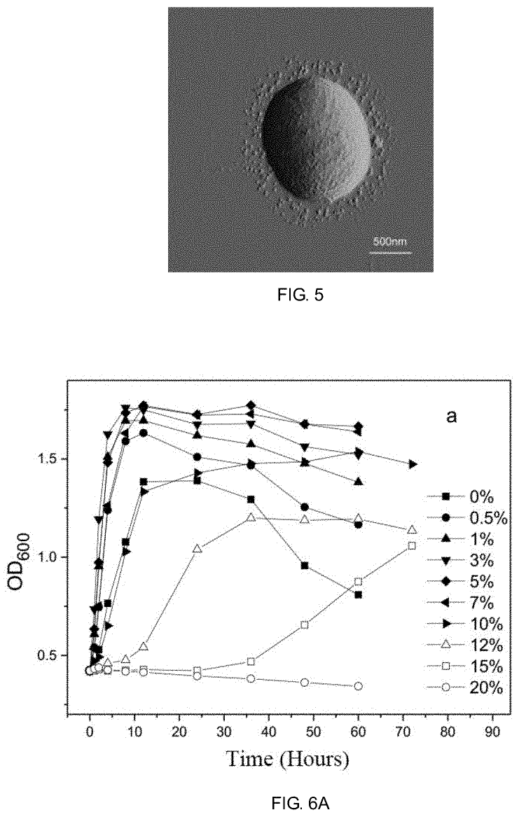

The main biological properties of the strain X3-1411 are: Gram-stain-negative (the result is shown in FIG. 4), observed to be bacillus with capsules but without flagella under an electronic microscope, which may be found singly, in pairs, or in short chains (the result is shown in FIG. 5). After 24 h culturing of the strain in the LB solid culture medium, the colony characteristic is characterized by round, smooth, and yellow.

The strain may grow in a culturing condition of 15° C.˜30° C., pH 7˜8, and salinity 0˜12% (best 1%˜5%).

2.3.2 Molecular Biological Identification:

The sequence of the 16s rDNA of the strain X3-1411 is shown in SEQ ID No. 3. By carrying out LBAS (web address: http://blast.ncbi.nlm.nih.gov/Blast.cgi) comparison between the sequence and that in the GenBank database, the result shows that the similarity between the sequence and the strain Erythrobacter citreus is 99.26%.

Based on the biological characteristic analysis of the strain and the 16s rDNA homology comparison result, the strain X3-1411 is identified as Erythrobacter citreus, which was collected in China Center for Type Culture Collection on Mar. 29, 2016 at Luojiashan, Wuchang, Wuhan City, with a collection number of CCTCC M 2016156. Example 2: Study on Phosphorous Removal Effects of the Strains of the

Present Application

1. Phosphorous Removal from Wastewater by the Strain Shewanella sp. CF8-6

Method of Applying the Strain Shewanella sp. CF8-6 in Water Treatment:

(1) Shewanella sp. CF8-6 was cultured in the seawater LB liquid culture medium for 24 hours under a condition of 25° C., 200 rpm, to prepare an activated bacteria solution.

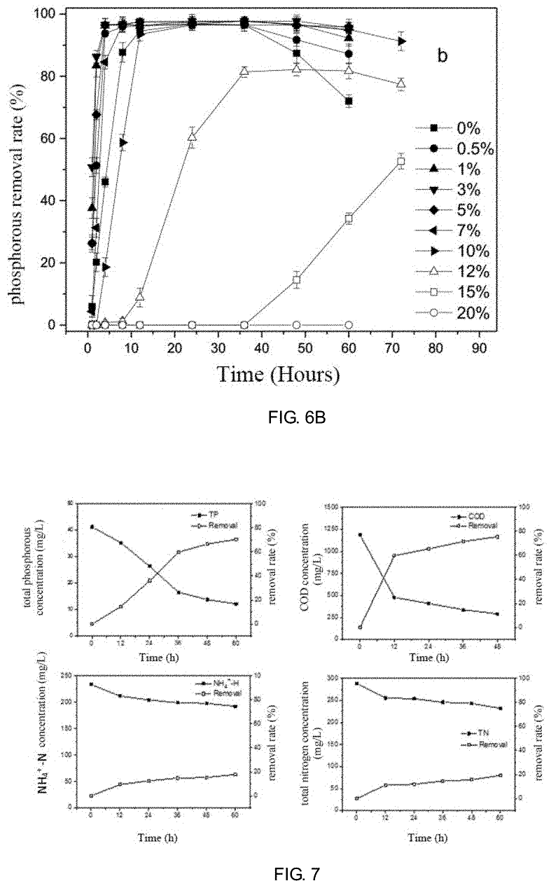

(2) The activated bacteria solution obtained in stage (1) was inoculated into simulated wastewater of different salinities (ranging from 0% to 20%) with a ratio of 10%. Samples ware cultured under a condition of 25° C. and 200 rpm. The concentrations of phosphorous in the supernatant and the biomass with the wavelength 600 nm were measured at different time points, to obtain phosphorous removal efficiencies and growth curves of the strain under different salinity ranges. The water treatment effects are shown in FIG. 6a and FIG. 6b.

From FIG. 6a and FIG. 6b, the strain Shewanella sp. CF8-6 of the present application has a high phosphorous removing efficiency in saline wastewater, particularly in the wastewater with a salinity of 10% or below. And the phosphorous removal rate within 10 hours may reach 99% above. Even in the wastewater with a salinity of 12% or 15%, the strain Shewanella sp. CF8-6 still has an excellent phosphorous removal rate.

Except the salinity, the simulated wastewater is coincident with the wastewater used in screening strains in components, where the phosphorous concentration (by P) is 10 mg/L.

2. Application of Psychrobacter aquimaris X3-1403 in Phosphorous-Contained Saline Wastewater Treatment

After cultured in LB for 24 hours, the Psychrobacter aquimaris X3-1403 was inoculated into the simulated seawater toilet-flushing wastewater with a ratio of 10%. And samples were taken by time points to measure the effects of the strain in TP, COD, NH₄⁺—N and TN removing. From FIG. 7, Psychrobacter aquimaris X3-1403 has a relatively high removal effect for TP and COD, with removal rates of 70.5% and 75.5%, respectively. And the TP and COD removal speeds at 48 hours are 0.57 mg/(L·h) and 18.7 mg/(L·h), respectively. However, this strain has a relatively poor removal effect for NH₄⁺—N and TN, with removal rates of 17.8% and 19.4%, respectively.

3. Phosphorous Removal Effect of Erythrobacter citreus X3-1411

(1) The Effect of the Erythrobacter citreus X3-1411 in Removing Phosphorous from Simulated Toilet-Flushing Wastewater

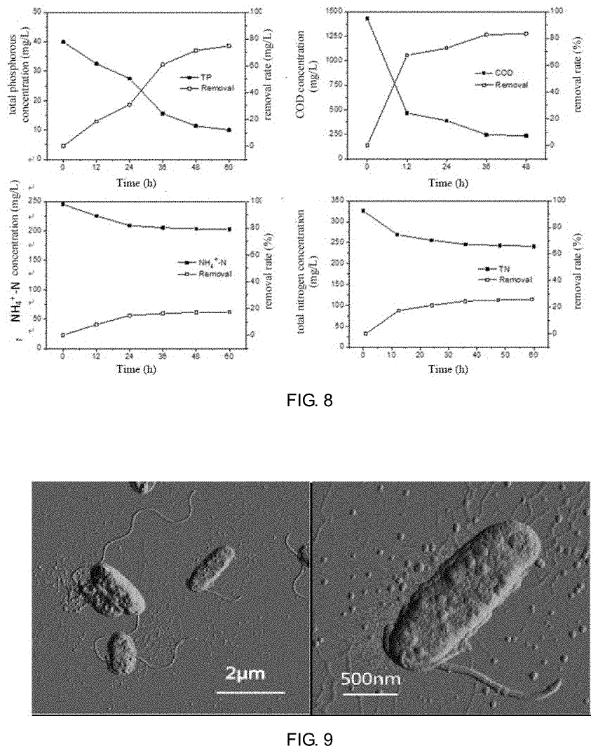

Erythrobacter citreus X3-1411, which was isolated and screened according to Example 1, was cultured in LB culture medium for 24 hours and then inoculated into the simulated seawater toilet-flushing wastewater with a ratio of 10%. The wastewater was cultivated at 25° C. at 200 rpm, and sampled by time points to measure the effects of the strain in removing TP, COD, NH₄⁺—N and TN. The results are shown in FIG. 8.

It may be seen from FIG. 8 that the strain has a relatively high removal effect for TP and COD, with the removal rates of 75.0% and 83.6%, respectively. And the TP and COD removal speeds at 48 hours are 0.59 mg/(L·h) and 24.9 mg/(L·h), respectively. However, this strain has a relatively poor removal effect for NH₄⁺—N and TN, for their removal rates being only 17.2% and 25.9%, respectively.

Components of the simulated seawater toilet-flushing wastewater in this example are coincident with that in Example 1.

(2) The Effect of Erythrobacter citreus X3-1411 in Removing Phosphorous from Simulated Domestic Wastewater

TABLE 2
Components of the Simulated Domestic Wastewater
(Mixed with Deionized Water):

| | Concentration | | Concentration |
|---|---|---|---|
| Components | (mg/L) | Components | (mg/L) |
| glucosum | 150 | NaCl | 500 |
| anhydricum | | | |
| Sodium acetate | 180 | CaCl₂ | 15 |
| Peptone | 75 | MgSO₄•7H₂O | 12.5 |
| Yeast extract | 50 | FeSO₄ | 0.3 |
| NH₄Cl | 100 | ZnSO₄•7H₂O | 0.1 |
| KH₂PO₄ | 20 | MnSO₄•7H₂O | 0.25 |
| Na₂HPO₄•12H₂O | 7.5 | CoCl₂•6H₂O | 0.025 |

### Example 3: Applications of the Strains of the Present Application in Preparing Self-Assembled Nanomaterial

1. Preparing a Nanomaterial by Shewanella sp. CF8-6

(1) Shewanella sp. CF8-6 was cultured in the seawater LB liquid culture medium for 24 hours under a condition of 25° C., 200 rpm, to prepare an activated bacteria solution. centrifugal parameters of the activated bacterial solution: centrifuging for 10 min at;

(2) After 10000 rpm centrifuged for 10 min and washed, the bacteria were inoculated at 10% (v/v) into the simulated wastewater. The bacteria were cultured for 48 hours at 25° C. and 200 rpm, and then centrifuged to obtain the bacteria cells, where the nanoparticles are distributed on the cell surfaces and their surroundings. The centrifugation is at 4000 rm, for 10 minutes.

(3) The bacteria cells containing nanoparticles obtained from stage (2) were washed twice with deionized water, at the rotation speed of 4000 rpm for 15 minutes. Then, the washed bacteria cells are observed by a TEM.

(4) After treated, the bacteria cell containing the nanoparticles obtained from stage (2) are observed for particle shape and size by an AFM.

Simulated High-Salinity Wastewater Formulation (1) (Low-Phosphorous Wastewater): C₆H₁₂O₆.H₂O 1.5 g/L, CH₃COONa 0.75 g/L, MgSO₄.7H₂O 1.18 g/L, NH₄Cl 0.9 g/L, KH₂PO₄.2H₂O 0.066 g/L (by P 10 mg/L:), NaCl 30 g/L, dissolved in tap water.

Simulated High-Salinity Wastewater Formulation (2) (High-Phosphorous Wastewater): C₆H₁₂O₆.H₂O 1.5 g/L, CH₃COONa 0.75 g/L, MgSO₄.7H₂O 1.18 g/L, NH₄Cl 0.9 g/L, glycerol phosphate disodium salt, (C₃H₆NaO₇P, by P: 50 mg/L), NaCl 30 g/L, CaCl₂ (by Ca: 80 mg/L), dissolved in deionized water.

The images of prepared nanomaterial are shown in FIG. 9 and FIGS. 10a-10f, respectively. FIG. 9 shows an image (AFM image) of the nanomaterial synthesized from the strain CF8-6 with simulated high-salinity wastewater Formulation (1). FIGS. 10a-10f show images of nanomaterial synthesized from the strain CF8-6 with simulated high-salinity wastewater Formulation (2), wherein FIG. 10a shows an AFM image; FIG. 10b and FIG. 10c show TEM images of nanoparticles; FIG. 10d, FIG. 10e, and FIG. 10f show TME images of self-assembled nanoparticle;

In the nanomaterials of the present application, the calcium phosphate nanoparticle has a particle size ranging from 100-200 nm.

2. Application of Psychrobacter aquimaris X3-1403 in Preparing Nano-Hydroxyapatite

The Psychrobacter aquimaris X3-1403 was activated in the LB culture medium for 24 hours under the culturing condition of 200 rpm, 25° C. Then, \textcircled{1} 25 mL activated culture solution was taken by using a pre-sterilized centrifugal tube in an aseptic operation table, and centrifuged at 10000 rpm for 10 min; \textcircled{2} the supernatant was removed, and the bacteria was re-suspended with 10 mL sterilized deionized water, and centrifuged at 10000 rpm for 10 min; \textcircled{3} stage \textcircled{2} was repeated once. The re-suspended bacterial solution was inoculated into the simulated seawater toilet-flushing wastewater (with a formula coincident with table 1) and the simulated domestic wastewater (with a formula identical to table 2) (with an inoculation amount of 10%). After cultured for 48 h at 200 rpm at 25° C., the culture solution was centrifuged at 5000 rpm for 10 min to remove the supernatant. The bacteria cells at the bottom of the centrifugal bottom were washed with deionized water to obtain the bacteria cells containing the nanomaterial. Part of the bacteria cells was re-suspended and fixed to a copper net, which was stained and finally dried for TEM observation.

The LB culture medium includes 1% peptone and 0.3% yeast, mixed with artificial seawater.

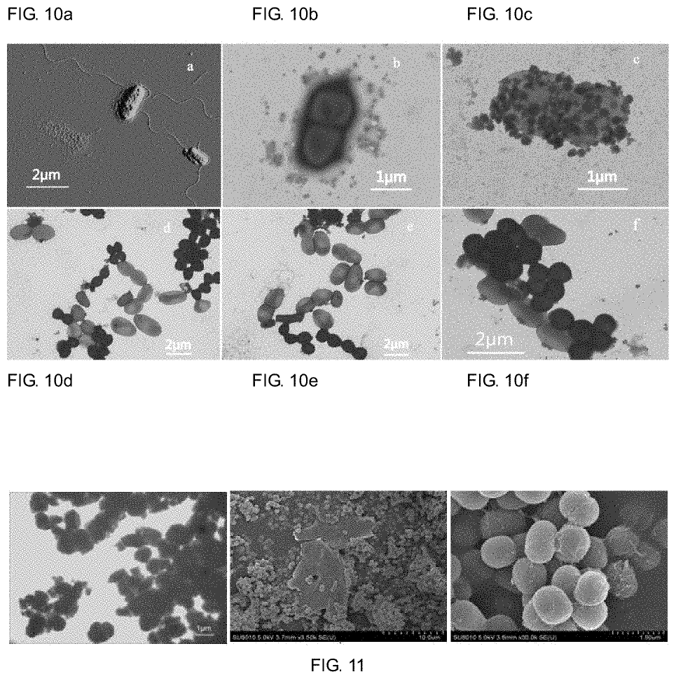

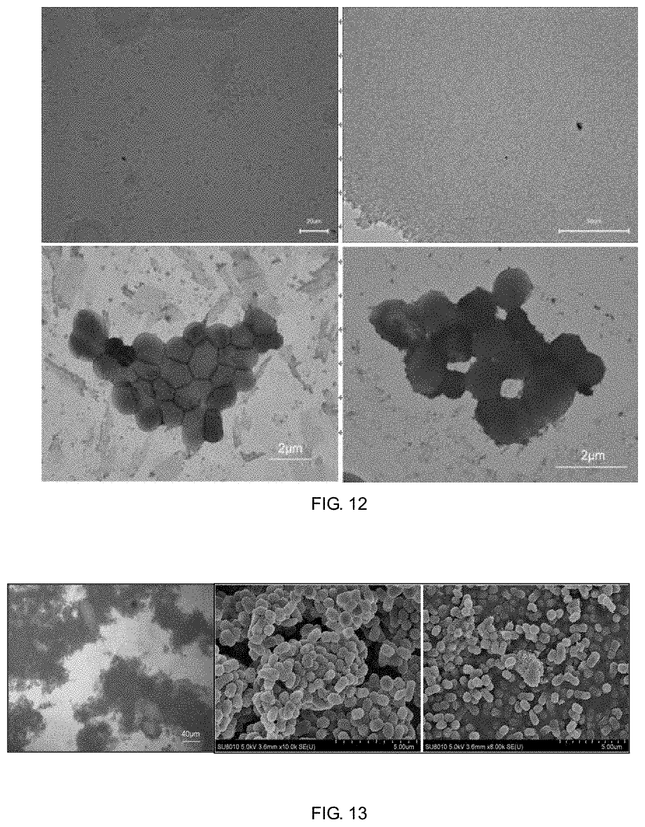

The electron microscope image of the self-flocculating bacteria cells in the simulated seawater toilet-flushing wastewater is shown in FIG. 11. The electron microscope image of the bacteria cells that synthesize and self-assemble the nanomaterial in the simulated seawater toilet-flushing wastewater is shown in FIG. 12. Both FIG. 11 and FIG. 12 show that the nanoparticle materials assume a honeycomb shape and a compact structure with uniformly distributed particle sizes at nanometer order; which are easily manufactured into a laminar nanomaterial.

3. Application of the Strain Erythrobacter citreus X3-1411 in Preparing Nanomaterials

Erythrobacter citreus X3-1411 was activated in the LB culture medium for 24 hours under a culturing condition of 200 rpm, at 25° C. Then, \textcircled{1} 25 mL activated culture solution was taken by using a pre-sterilized centrifugal tube in an aseptic operation table and centrifuged at 10000 rpm for 10 min; \textcircled{2} the supernatant was removed, and the bacteria solution was re-suspended with 10 mL sterilized deionized water, and centrifuged at 10000 rpm for 10 min; \textcircled{3} stage \textcircled{2} was repeated once. The re-suspended bacterial solution was inoculated into the simulated seawater toilet-flushing wastewater (with a formula coincident with table 1) and the simulated domestic wastewater (with a formula coincident with table 2) (with an inoculation amount of 10%). After cultured for 48 h at 200 rpm at 25° C., the culture solution was centrifuged at 5000 rpm for 10 min to remove the supernatant. The bacteria cells at the bottom of the centrifugal bottom were washed with the deionized water to obtain the bacteria cells containing the nanomaterial. Part of the bacteria cells was re-suspended and fixed to the copper net, which was stained, and finally dried for TEM observation.

The LB culture medium includes 1% peptone and 0.3% yeast, mixed with artificial seawater.

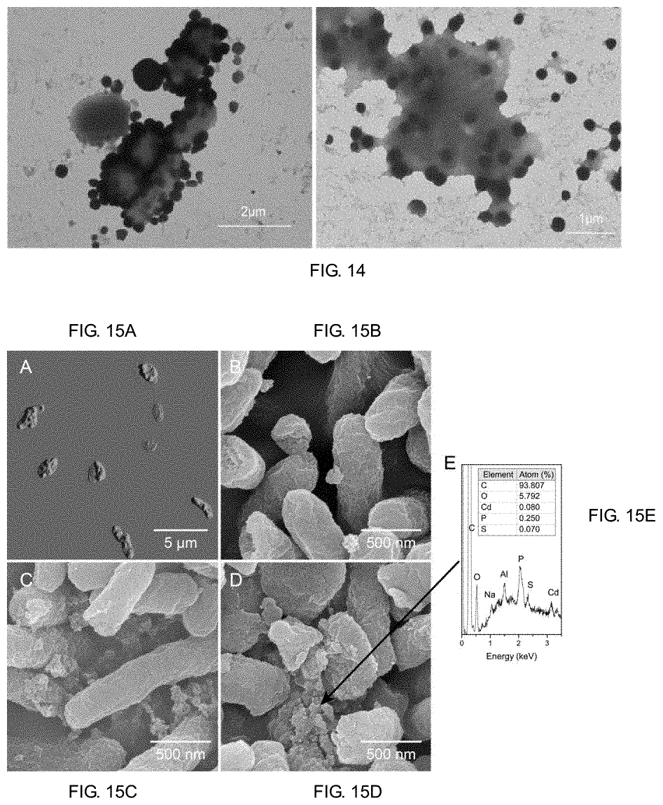

The electron microscope image of the self-flocculating bacteria cells in the simulated seawater toilet-flushing wastewater is shown in FIG. 13. The TEM image of the bacteria cells that synthesize and self-assemble the nanomaterial in the simulated seawater toilet-flushing wastewater is shown in FIG. 14. Both FIG. 13 and FIG. 14 show that the material has a good dispersion and a uniform particle size distribution.

4. Application of the Pseudoalteromonas sp. DSBS for Preparing a Nanomaterial

(1) Application of the Pseudoalteromonas sp. DSBS for Preparing a Self-Assembled Nanomaterial in a Low-Salinity Wastewater

Pseudoalteromonas sp. DSBS was inoculated at 0.9% (v/v) in a liquid LB culture medium and cultured in a thermostatic shaker at 25° C. and 200 rpm for 20 h to obtaini enriched bacterial cells.

The enriched bacteria suspension solution was inoculated at 10% (v/v) into the low-salinity wastewater. And the wastewater was cultured in a thermostatic shaker for 48 h at 25° C. and 200 rpm to obtain a bacteria suspension solution containing cadmium-phosphorous-sulfur nanoparticles.

The bacteria suspension solution containing cadmium-phosphorous-sulfur nanoparticles was centrifuged and washed with deionized water to obtain the nanomaterial. The centrifuging speed was 3000 rpm to prevent washing off the particles on the bacteria cell surfaces.

Components of the liquid LB culture medium are provided below:

Peptone 10 g/L, yeast extract 3 g/L, mixed with artificial seawater, where the seawater salinity is 3.5%.

Components of the low-salinity wastewater are provided below:

D-Glucose monohydrate 5.06 g/L, NaAC 1.5 g/L, NaCl 3.5 g/L, NH₄Cl 2.6 g/L, MgSO₄.7H₂O 2.4 g/L, wherein the total cadmium content (Cd(NO₃)₂.4H₂O) and total phosphorous content (K₂HPO₄) are 8 mg/L and 9 mg/L, respectively; the initial pH is 7.2 and the salinity is 0.35%. To simulate the low-salinity wastewater environment, the cadmium concentration and phosphorous concentration refer to their concentrations in general industrial cadmium-contained wastewater, which does not suffice to form inorganic chemical cadmium precipitations with this pH and ion concentrations. Therefore, this environment belongs to a cadmium-phosphorous μM-order unsaturated low-salinity environment.

To further characterize the bacteria cells and the formed nanoparticles, the cultured bacteria cells after removal of phosphorous and cadmium are observed with an atomic force microscope (AFM) and a scanning electron microscope (SEM). The observation result of the AFM is shown in FIG. 15A, where nanoparticle substances with diameters ranging from 25 to 100 nm are uniformly aggregated on the bacteria cell surfaces and scattered around. In the figure, some bacteria cells have flagella, while some do not, which are cast off during culture or sample pre-treatment.

During the pre-treatment process of the bacteria cells for the SEM, the centrifugal speed for washing with a phosphate buffer is 3000 rpm to avoid washing off the particles on the bacteria cell surfaces, while the centrifugal speed for other processes is 6000 rpm. The results are shown in FIGS. 15B, 15C, and 15D. In FIG. 15B, smaller particles with diameters ranging from 25 to 60 nm are aggregated into a uniform sphere with a diameter of 100 nm, which is attached to the bacteria cell surfaces. In FIG. 15C, smaller particles are aggregated on fibers of exopolysaccharide. In FIG. 15D, smaller particles are agglomerated between bacterial cells. This indicates that the nanoparticles synthesized by the bacteria have three different morphologies: small particles with diameters ranging from 25 nm to 60 nm uniformly dispersed on bacteria cell surfaces, small particle spherical aggregations with a diameter of 100 nm attached onto the bacteria cell surfaces, and the aggregates being cast off from the bacteria and agglomerated and attached on exopolysaccharide fibers between the bacteria cells. Meanwhile, EDS (Energy Dispersive Spectrometer) analysis of the point locations in the figures shows that the particles mainly contain C, O, Cd, P, and S elements. The nanoparticles are cadmium-phosphorous-sulfur nanoparticles mixed with polysaccharide, where Na refers to the precipitated of dissolvable ion and Al refers to a sample stage element, neither of which are elements in the nanoparticles.

Therefore, the bacteria realized simultaneous removal of phosphorous and cadmium in low-salinity wastewater with unsaturated phosphorous and cadmium concentrations, and three morphologies of cadmium-phosphorous-sulfur nanoparticle mixed with polysaccharide (with a diameter ranging from 25-60 nm) are extracellularly formed.

(2) Application of the Pseudoalteromonas sp. DSBS for Preparing a Nanomaterial in High-Salinity Wastewater

Pseudoalteromonas sp. DSBS was inoculated at 0.9% (v/v) in a liquid LB culture medium, and cultured in a thermostatic shaker at 25° C. and 200 rpm for 20 h to obtain enriched bacterial cells.

The enriched bacteria cells were centrifuged at 3000 rpm for 10 min to remove the supernatant. The bacteria were re-suspended to the original volume with high-salinity wastewater, to obtain the bacteria cells washed once.

The washed bacteria suspension solution was inoculated with 0.2% (v/v) into three groups of high-salinity wastewater. The wastewater was cultured in a thermostatic shaker for 48 h at 25° C. and 200 rpm to obtain a bacteria suspension solution containing cadmium-phosphorous-sulfur nanoparticles.

The bacteria suspension solution containing cadmium-phosphorous-sulfur nanoparticles was washed with deionized water at a centrifuging speed of 3000 rpm to prevent washing off the particles on the bacteria cell surfaces, thereby obtaining the nanomaterial.

Preferably of the present application, components of the liquid LB culture medium are provided below:

Peptone 10 g/L, yeast extract 3 g/L, mixed with artificial seawater, where the seawater salinity is 3.5%.

Components of the high-salinity wastewater are provided below:

NaAC 0.82 g/L, NH₄Cl 0.11 g/L, sea salt 33.33 g/L, initial pH 7.2, salinity 3.5%. The three groups of synthesized seawater differ in total cadmium (Cd(NO₃)₂.4H₂O) concentration and total phosphorous concentration (K₂HPO₄), which are 0.1756×10⁻³ mg/L and 0.1548 mg/L for group A, 5.671×10⁻³ mg/L and 5 mg/L for group B, and 10.21×10⁻³ mg/L and 9 mg/L for group C. To simulate the high-salinity wastewater environment, the cadmium concentration and phosphorous concentration refer to their concentrations in seawater, which belongs to a cadmium nM-order unsaturated high-salinity environment.

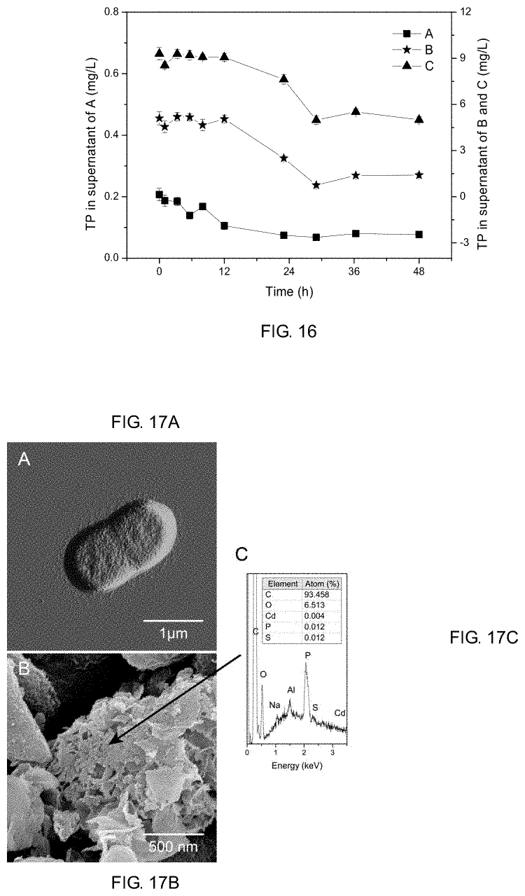

Among the three groups of wastewaters, changes of the total phosphorous concentration in the supernatant are measured by using the ammonium molybdate spectrophotometric process; the results are shown in FIG. 16. In a high-salinity environment, this strain has a 46.24% removal rate for 9 mg/L phosphorous and a 72.48% removal rate for 5 mg/L phosphorous.

To further characterize the bacteria cells and the formed nanoparticles, the bacteria cells are observed with the AFM and the SEM. The results of AFM are shown in 17 A, where nanoparticle with the diameters under 10 nm are uniformly dispersed on the bacteria cell surfaces. In the synthesized seawater, the concentrations of the phosphorous and cadmium are at nM-order. And a lower cadmium concentration causes a smaller particle size of the nanoparticles. Additionally, in this test, the bacteria cells have no flagella, which might be unnoticeable due to rupture.

The result of SEM is shown in FIG. 17 B. Spherical particles with a diameter of 10 nm are existent between bacteria cells, which are attached to the fiber-shaped exopolysaccharide. Meanwhile, EDS (Energy Dispersive Spectrometer) (FIG. 17 C) analysis of the point locations in the figures shows that the particles also mainly contain C, O, Cd, P, and S elements.

Therefore, Pseudoalteromonas sp. DSBS also form cadmium-phosphorous-sulfur nanoparticles mixed with polysaccharide (with a diameter of 10 nm) extracellularly in the high-salinity wastewater with unsaturated phosphorous and cadmium concentrations.

What have been described above are only preferred embodiments of the present application, not for limiting the present application; to those skilled in the art, the present application may have various alterations and changes. Any modifications, equivalent substitutions, and improvements within the spirit and principle of the present application should be included within the protection scope of the present application.

The invention also relates to a class of aerobic efficient-phosphorus-removal bacteria that enable to biologically self-assemble and synthesize nanoparticles while wastewater treatment, include Shewanella sp. CF8-6, Psychrobacter aquimaris X3-1403, and Erythrobacter citreus X3-1411, among which:

the Shewanella sp. CF8-6 was collected in China Center for Type Culture Collection on Mar. 29, 2016 at Luojiashan, Wuchang, Wuhan City, with a collection number of CCTCC M 2016154;

the Psychrobacter aquimaris X3-1403 was collected in China Center for Type Culture Collection on Mar. 29, 2016 at Luojiashan, Wuchang, Wuhan City, with a collection number of CCTCC M 2016155; and

the Erythrobacter citreus X3-1411 was collected in China Center for Type Culture Collection on Mar. 29, 2016 at Luojiashan, Wuchang, Wuhan City, with a collection number of CCTCC M 2016156.

The invention also relates to a microbial agent with an active ingredient selected from at least one bacterium (Shewanella sp. CF8-6, Psychrobacter aquimaris X3-1403, and Erythrobacter citreus X3-1411) as disclosed above.

According to a further development, the microbial agent includes a solid of liquid carrier.

According to a further development, the active ingredient is the cultured living cell, a fermentation broth of the living cell, a filtrate of a cell culture, or a mixture of cell and filtrate.

According to a further development, a dosage form of the microbial agent is liquor, suspension concentrate, powder, granules, wettable powder, or water dispersible granules.

The invention also relates to a biofilm or a biofilm reactor including at least one strain selected from the group consisting of Shewanella sp. CF8-6, Psychrobacter aquimaris X3-1403, and Erythrobacter citreus X3-1411 as disclosed above.

The invention also relates to an application of the bacterial strains as disclosed above, the microbial agent as disclosed above, and/or the biofilmor biofilm reactor as disclosed above to remove phosphorous from wastewater.

The invention also relates to a process of removing phosphorous from saline wastewater, comprising steps of:

inoculating a microbial strain as disclosed above to an LB culture medium, culturing it to prepare an activated bacteria solution, and adding the activated bacteria solution at an 8-12% volume fraction into a to-be-treated wastewater;

or adding the microbial agent as disclosed above into the to-be-treated wastewater.

The invention also relates to an application of the strains as disclosed above and/or the microbial agent as disclosed above in preparing a sewage treatment agent.

The invention also relates to an application of a strain as disclosed above or Pseudoalteromonas sp. DSBS with a collection number of CCTCC M2013652 in preparing a self-assembly nanomaterial.

The invention also relates to a self-assembled biological nanomaterial, which is synthesized by a strain as disclosed above or Pseudoalteromonas sp. DSBS with a collection number of CCTCC M2013652 in phosphorous-containing wastewater and prepared through self-assembly.

According to a further development, a concentration of phosphorous in the phosphorous-contained wastewater is 0.3 mM-1.3 mM.

The invention also relates to a preparing process of the self-assembled biological nanomaterial as disclosed above, comprising a stage of a strain activating, and a stage of the activated strain culturing and self-assembling in a phosphorous-contained wastewater.

According to a further development, the stage of culturing and self-assembling further comprises: centrifuging a cultured solution to remove a supernatant, and obtaining bacteria cells containing nanomaterial, namely the biological nanomaterial.

The invention also relates to the use of the microbial self-assembled nanomaterial as disclosed above, comprising:

(1) application in heavy metal pollution remediation;

or (2) application in preparing a carrier or material utilized for a biomedicine.

The invention also relates to a preparing method of nano-hydroxyapatite, wherein the nano-hydroxyapatite is obtained by purifying and isolating the microbial self-assembled nanomaterial as disclosed above to remove organics.

According to a further development, a method adopted by the purification and isolation is calcining the microbial self-assembled nanomaterial.

1-7. (canceled)

8. A process of removing phosphorous from saline wastewater, comprising the steps of:
inoculating a microbial strain into a to-be-treated wastewater, wherein the microbial strain comprises at least one strain selected from the group consisting of: Shewanella sp. CF8-6, Psychrobacter aquimaris X3-1403, and Erythrobacter citreus X3-1411, wherein;
the Shewanella sp. CF8-6 was collected in China Center for Type Culture Collection on Mar. 29, 2016 at Luojiashan, Wuchang, Wuhan City, with a collection number of CCTCC M 2016154,
the Psychrobacter aquimaris X3-1403 was collected in China Center for Type Culture Collection on Mar. 29, 2016 at Luojiashan, Wuchang, Wuhan City, with a collection number of CCTCC M 2016155, and
the Erythrobacter citreus X3-1411 was collected in China Center for Type Culture Collection on Mar. 29, 2016 at Luojiashan, Wuchang, Wuhan City, with a collection number of CCTCC M 2016156.

9-17. (canceled)

18. The process of claim 8, further comprising: culturing the microbial strain in an LB culture medium at about 0-15% salinity to prepare an activated bacteria solution, and adding the activated bacteria solution to the to-be-treated wastewater.

19. The process of claim 8, wherein the microbial strain is an aerobic efficient-phosphorus-removal bacteria that can, using wastewater, biologically self-assemble into nanoparticles.

20. The process of claim 8, wherein the microbial strain is added to the wastewater at a concentration of about 8 to 12% volume.

21. The process of claim 8, wherein the microbial strain is added to the wastewater at a concentration of about 5 to 20 mg/L.

22. The process of claim 18, wherein the LB medium comprises 1% peptone, 0.3% yeast mixed with artificial seawater (0-15% dissolved salts).

23. The process of claim 18, wherein the microbial strain is cultured for 18 to 30 hours to prepare the activated bacteria solution.

24. The process of claim 18, wherein the microbial strain is cultured for a time sufficient to synthesize nanoparticles by microbial self-assembly.

25. The process of claim 8, wherein the microbial strain further comprises a solid or liquid carrier.

26. The process of claim 8, wherein the microbial strain is selected from a cultured living cell, a fermentation broth of the living cell, a filtrate of a cell culture, or a mixture of cell and filtrate.

27. The process of claim 8, wherein the microbial strain is added to the wastewater as a liquor, suspension concentrate, powder, granule, wettable powder, or water dispersible granule.

28. The process of claim 8, wherein the microbial strain is inoculated into the wastewater at a concentration sufficient to remove phosporous from the wastewater.

29. The process of claim 8, further comprising culturing the microbial strain in the wastewater for a time sufficient to remove phosphorous from the wastewater.


SEQUENCE LISTING (3 sequences)

SEQ ID NO: 1 - DNA, 1492 bp, Shewanella sp.
tgatcatggctcagattgaacgctggcggcaggcctaacacatgcaagtcgagcggaaacacaagggagcttgctcctgaggtgtcgagcggcggacgggtgagtaatacctaggtatctgcccaatcgagggggataacagttggaaacgactgctaataccgcatacgccctacgggggaaaggaggggaccttcgggcctttcgcgattggatgaacctaggcgggattagctagttggtgaggtaatggctcaccaaggcgacgatccctagctggtctgagaggatgatcagccacactggaactgagacacggtccagactcctacgggaggcagcagtggggaatattgcacaatgggcgaaagcctgatgcagccatgccgcgtgtatgaagaaggccttcgggttgtaaagtactttcagcgaggaggaaaggttgttggttaataaccaacagctgtgacgttactcgcagaagaagcaccggctaacttcgtgccagcagccgcggtaatacgaggggtgcaagcgttaatcggaattactgggcgtaaagcgtacgcaggcggtttgttaagcgagatgtgaaagccccgggctcaacctgggaactgcatttcgaactggcaaactagagtcttgtagaggggggtagaatttcaggtgtagcggtgaaatgcgtagagatctgaaggaataccggtggcgaaggcggccccctggacaaagactgacgctcatgtacgaaagcgtggggagcaaacaggattagataccctggtagtccacgccgtaaacgatgtctactcggaatttggtgtcttgaacactgggttctcaagctaacgcattaagtagaccgcctggggagtacggccgcaaggttaaaactcaaatgaattgacgggggcccgcacaagcggtggagcatgtggtttaattcgatgcaacgcgaagaaccttacctactcttgacatccagagaattcgctagagatagcttagtgccttcgggaactctgagacaggtgctgcatggctgtcgtcagctcgtgttgtgaaatgttgggttaagtcccgcaacgagcgcaacccttatccttatttgccagcacgtaatggtgggaactttagggagactgccggtgataaaccggaggaaggtggggacgacgtcaagtcatcatggcccttacgagtagggctacacacgtgctacaatggccggtacagagggttgcaaagccgcgaggtggagctaatctcacaaagccggtcgtagtccggatcggagtctgcaactcgactccgtgaagtcggaatcgctagtaatcgtgaatcagaatgtcacggtgaatacgttcccgggccttgtacacaccgcccgtcacaccatgggagtgggctgcaccagaagtagatagcttaaccttcgggagggcgtttaccacggtgtggttcatgactggggtgaagtcgtaacaag

SEQ ID NO: 2 - DNA, 1405 bp, Psychrobacter aquimaris  X3-1403
gtgacgcctccccgaaggttaagctatccacttctggtgcaatcaactcccatggtgtgacgggcggtgtgtacaaggcccgggaacgtattcaccgcggcattctgatccgcgattactagcgattcctacttcatggagtcgagttgcagactccaatctggactacgataggctttttgagattcgcatcacatcgctgtgtagctgccctctgtacctaccattgtagcacgtgtgtagccctggtcgtaagggccatgatgacttgacgtcgtccccgccttcctccagtttgtcactggcagtatccttagagttcccggcttaacccgctggtaactaaggacaagggttgcgctcgttgcgggacttaacccaacatctcacgacacgagctgacgacagccatgcagcacctgtattctaattcccgaaggcactcccgcatctctgcaggattctagatatgtcaagaccaggtaaggttcttcgcgttgcatcgaattaaaccacatgctccaccgcttgtgcgggcccccgtcaattcatttgagttttaaccttgcggccgtactccccaggcggtctacttattgcgttagctgcgtcactaagtcctcaagggacccaacgactagtagacatcgtttacggcgtggactaccagggtatctaatcctgtttgctacccacgctttcgagcctcagtgtcagtatgatgccaggaagctgccttcgccatcggtattccttcagatctctacgcatttcaccgctacacctgaaattctacttccctctcacctactctagcctaacagtttcagatgcagttcccaggttaagcccggggatttcacatctgacttatcaagccacctacgctcgctttacgcccagtaattccgattaacgcttgcaccctctgtattaccgcggctgctggcacagagttagccggtgcttattctgcagctaatgtcatcgtccgtgggtattaaccacggagtcttcttcactgcttaaagtgctttacaaccaaaaggccttcttcacacacgcggcatggctggatcagggtttcccccattgtccaatattccccactgctgcctcccgtaggagtccgggccgtgtctcagtcccggtgtggctgatcatcctctcagaccagctacagatcgtcgccatggtaggcctttaccccaccatctagctaatccgacttaggctcatctaatagcgagagcagtaaactgccccctttctcccgtaggtcgtatgcggtattaatacgagtttccccgtgctatcccccactactaggtagattcctaagtattactcacccgtccgccgctcgacgcctggtagcaagctaccatcgttccgctcgactgca

SEQ ID NO: 3 - DNA, 1354 bp, Erythrobacter citreus  X3-1411
gtcggctgcctcctaaaggttagcgcaccgccttcgggtgaatccaactcccatggtgtgacgggcggtgtgtacaaggcctgggaacgtattcaccgcggcatgctgatccgcgattactagcgattccgccttcatgctctcgagttgcagagaacaatccgaactgagacatcttttggagattagctaaccctcgcgggatcgctgctcactgtagatgccattgtagcacgtgtgtagcccagcctgtaagggccatgaggacttgacgtcatccccaccttcctccggcttatcaccggcagtttccttaaagtgcccaactaaatgatggcaactaaggacgagggttgcgctcgttgcgggacttaacccaacatctcacgacacgagctgacgacagccatgcagcacctgtcactaggtccccgaagggaagaaatctgtctccagaagtcgtcctaggatgtcaaaggctggtaaggttctgcgcgttgcttcgaattaaaccacatgctccaccgcttgtgcaggcccccgtcaattcctttgagttttaatcttgcgaccgtactccccaggcggataacttaatgcgttagctgcgccacccaagctccatgagcccggacagctagttatcatcgtttacggcgtggactaccagggtatctaatcctgtttgctccccacgctttcgcacctcagcgtcaataactgtccagtgagtcgccttcgccactggtgttcttccgaatatctacgaatttcacctctacactcggaattccactcacctctccagtattctagccatccagtttcaagggcagttccggggttgagccccgggatttcacccctgacttgaaaagccgcctacgtgcgctttacgcccagtaattccgaacaacgctagctccctccgtattaccgcggctgctggcacggagttagccggagcttattctccaggtactgtcattatcatccctggtaaaagagctttacaaccctaaggccttcatcactcacgcggcattgctggatcaggctttcgcccattgtccaatattccccactgctgcctcccgtaggagtctgggccgtgtctcagtcccagtgtggctgatcatcctctcagaccagctatggatcgtcgacttggtaggccattaccccaccaactatctaatccaacgcgggcccatctaaaggcaataaatctttggtccgaagacattatccggtattagcagtcatttctaactgttattccgaacctaaaggcaggttcccacgcgttacgcacccgtgcgccactaaccccgaagggttcgttcgacttgca